(19) United States
(12) Patent Application Publication
(10) Pub. No.: US 2007/0219412 A1
(43) Pub. Date: Sep. 20, 2007

(54) Flexible arthroscope and method of using the same

(76) Inventors: Christopher William DiGiovanni, Barrington, RI (US); Stephen L. Tocci, Warwick, RI (US); Amar Patel, Cranston, RI (US)

Correspondence Address: SKOKOS LAW GROUP, LLC, 1100 JORIE BOULEVARD, SUITE 220, OAK BROOK, IL, 60523, US

(21) Appl. No.: 11/725,102

(22) Filed: Mar. 16, 2007

Related U.S. Application Data
(60) Provisional application No. 60/783,348, filed on Mar. 18, 2006.

(51) Int. Cl. A61B 1/00; A61B 1/12

(52) U.S. Cl. 600/146; 600/160; 600/142; 600/156; 600/109

(57) ABSTRACT

A flexible arthroscope comprising a semi-flexible examination tube having means for bending at its distal end, and removably connected to a handheld module at its proximal end. The semi-flexible examination tube coupled with its means for bending its distal end provides the user the ability to immediately alter his field of view in any direction and maintain the horizontal plane.

### CROSS REFERENCE TO RELATED APPLICATION

The present application is a regular utility application claiming the benefits of the filing date of provisional application, Application No. 60/783,348, filed on Mar. 18, 2006 by the present applicants.

### BACKGROUND OF THE INVENTION

The present invention relates generally to arthroscopes, endoscopes and similar optical instruments.

While current devices exist that may be used for inspecting the interior of a human joint (such as the ankle, hip, knee, elbow, wrist etc), these devices do not provide for any significant flexibility and thereby limit the visual field available to the surgeon. This current “rigid design” technology has historically been very effective for basic arthroscopic procedures in the larger joints of the musculoskeletal system over the past few decades. However, as orthopaedic surgery has continued to evolve into trying to accomplish “more” through “less” and expand the surgical indications for what can be accomplished, do so over a broader range and greater complexity of joints and continue to do this through smaller incisions and hence lower risk, this rigid arthroscopic technology has lost pace with the demand requirements of the field. In short, decade's old technology is being applied to more complex state of the art procedures and more intricate joints, proving to be inadequate. Current designs have not followed function/application, and this continues to increasingly force the orthopaedic surgeon to rely on adjuvant and potentially risky inadequate methods during surgery to perform more complicated procedures, which can lead to: 1) iatrogenic articular damage (scuffing of the joint surface when trying to place a straight object across a curved joint), 2) inadvertent overdistraction of the joint (excessive tissue pull to enable a wider space for insertion of the instrument), 3) the inconvenient, expensive and time consuming need to alternate between multiple arthroscopes with differing viewing angles that trade, rather than complement, one view for another, 4) reliance on a poor visual field through which to perform one's surgery or acceptance of suboptimal intervention for a given procedure, or 5) increased portal creation or conversion to an open procedure (hence increasing risk or defeating the original purpose of the arthroscopy) to complete the procedure.

Devices with limited flexibility are currently in use today, but have evolved for general (not orthopaedic) surgical indications, hence being practically designed as endoscopic (not arthroscopic) instruments meant to cannulate and maneuver through soft tissue lumens in areas such the gastrointestinal tract, genitourinary system, and respiratory system. Although these devices are flexible, they lack the alternative utilities of the current rigid orthopaedic arthroscopes for use in the inflexible architecture of our musculoskeletal system and joints. Such devices do not provide for proper length or diameter for joint evaluation, lack means for proper water flow to enable joint distention and flushing, are cumbersome for arthroscopic surgery, lack the appropriate portal accessibility necessary for orthopaedic procedures and are perhaps not ideally designed nor suited for the repetitive absolute sterilization required for a routine day of arthroscopic surgery (not similarly required at that same level of sterilization for GI colonoscopy for example).

The present invention overcomes these obstacles and represents an attempt to combine the advantages of both technologies to improve and advance arthroscopic orthopaedic surgery specifically by providing a flexible arthroscope for both small (ex., elbow, ankle, subtalar, and wrist) and large (ex., hip, knee, shoulder) joint evaluation, providing adequate flow, visualization, trans-instrument manipulation in a size and length appropriate for small or large joint cannulation, and direction of line of sight. Most, if not all current commercially available endoscopes flex in one plane only and rotate both the horizon and visual field together. The present invention would enable movement in two orthogonal planes at the tip and also enable rotation of the tip without changing the “horizon” such that the surgeon would not lose his or her field of reference during arthroscopy with this instrument.

### SUMMARY OF THE INVENTION

Briefly described, the present invention, in one embodiment, is directed to a flexible arthroscope comprising a semi-flexible examination tube having means for bending in at least one plane at its distal end, and removably connected to a handheld module at its proximal end. The semi-flexible examination tube coupled with its means for bending its distal end provides the user the ability to immediately alter his field of view in any direction and maintain the horizontal plane. The handheld module being removably connected to the semi flexible examination tube enables the user to separate the handheld module from the semi-flexible examination tube such that 1) they can be sterilized independent of one another or replaced independent of another if necessary, and 2) to allow a user to replace the semi-flexible examination tube with one of another size or length intraoperatively if needed for surgical purposes or if the semi-flexible examination tube becomes contaminated while the handheld module remained sterile.

In one embodiment of the present invention, the semi-flexible examination tube may be of a durable, semi-flexible plastic capable of sterilization. The distal portion of the semi-flexible examination tube contains means for bending which may include intercalated segments having the ability to bend in an arc ranging up to 180 degrees. The intercalated segments may be coupled to a pulley system that is controlled via a lever or rotary knob in the handheld module.

### BRIEF DESCRIPTION OF THE DRAWINGS

FIG. 1 is a perspective, exploded view of a flexible arthroscope designed in accordance with an embodiment of the present invention.

### DETAILED DESCRIPTION OF THE EMBODIMENTS

The present invention, in one embodiment, is directed to a flexible arthroscope comprising a semi-flexible examination tube having means for bending at its distal end, and removably connected to a handheld module at its proximal end.

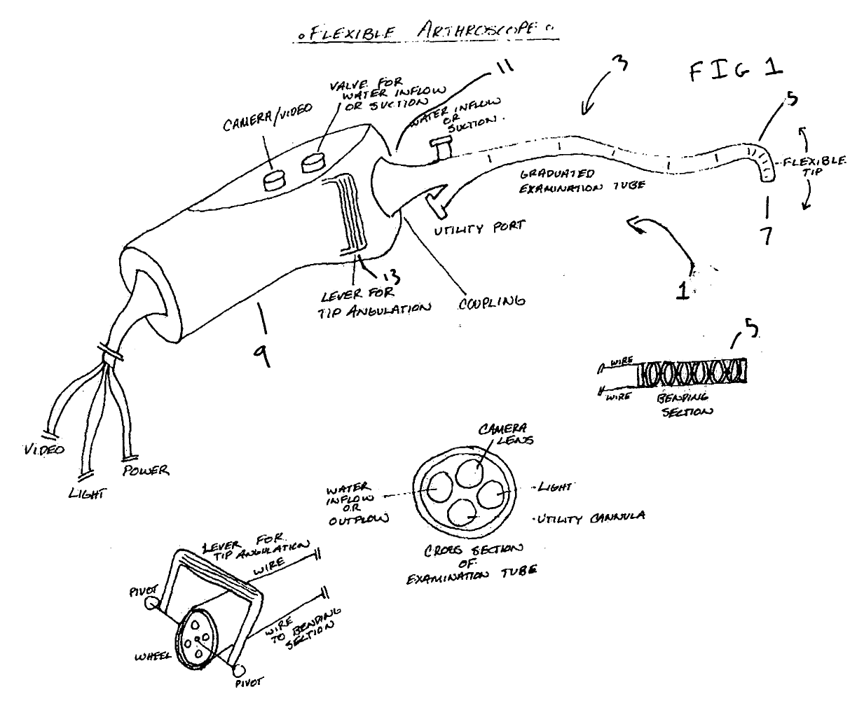

FIG. 1 illustrates an embodiment of a flexible arthroscope 1 wherein a semi-flexible examination tube 3 having means for bending 5 at its distal end 7 is removably connected to a handheld module 9 at its proximal end 11. The distal end 7 of the semi flexible examination tube 3 contains means for bending 5 which as shown in FIG. 1 may include intercalated segments having the ability to bend in an arc of up to 180 degrees from the longitudinal axis of the semi-flexible examination tube 3. The intercalated segment are segments of the semi flexible examination tube 3 that are linked together such that each segment is allowed to bend and move according to the movement of its adjacent segment, thereby creating a bending mechanism in the distal end 7 of the semi flexible examination tube 3. Preferably, the intercalated segments may be coupled to a pulley system that is controlled via a lever and/or rotary knob 13 in the handheld module 9. The pulley system includes wires that are connected at one end to the lever or rotary knob 13 and travel along the length of the semi flexible examination tube 3 to connect at the opposite end to the intercalated segments. The pulley system provides the user with control over the bending of the intercalated segments. The means for bending may be intercalated segments as shown or may be segments that are linked together via pins, wire reinforced tubing, segments connected via spring means, inflatable tubing or any other conventional means for bending.

In the present invention, the semi-flexible examination tube 3 may be proportionate to the amount needed to successfully cannulate the interstices of individual joints. Variation in the size of the diameter and length of the semi-flexible examination tube would allow access to either large or small, superficial or deep joints of the musculoskeletal system. The semi-rigid portion of the semi-flexible examination tube 3 between the proximal end 11 and means for bending 5 would be flexible enough to negotiate anatomic constraints in each joint. Since none of these joints are linear in nature and many are difficult to distract (to widen the available space for a straight, rigid cannula) or negotiate due to anatomic complexity (worse than the knee, which represents the primary joint for which orthopaedic arthroscopy has developed and is easier to cannulate and negotiate as compared to most other joints now also arthroscopically evaluated) this flexibility offers a significant advantage over current devices. The semi-flexible examination tube 3 may vary in length and diameter to satisfy the anatomic requirements of each joint and is interchangeable with other sizes, lengths, or diameters, depending on joint requirements. In the present invention, a cannula may be used to both insert and withdraw the flexible arthroscope 1.

In a preferred embodiment, as shown in FIG. 1, a collinear knob or lever mechanism would control the flexibility of the means for bending 5 of the semi-flexible examination tube 3. Since the means for bending 5 would move collinear with the lever or knob, the surgeon would always know how to hold the handheld module 9 in order to properly control and negotiate the means for bending 5 within the confines of the joint.

In a preferred embodiment, the flexible arthroscope 1 has lighting means at the distal end 7 of the semi-flexible examination tube 3. The lighting means may include optical fibers connected at one end to an external light source and at the opposite end travel lengthwise along the semi-flexible examination tube 3 to an opening at the distal end of the semi-flexible examination tube 3 in order to illuminate the joint for the user.

In a preferred embodiment, the flexible arthroscope 1 has still image and video capture means. A camera lens may be coupled to optical fibers, or alternatively a small camera or camera lens may be placed at the distal end of the semi-flexible examination tube 3, for image or video capture. The still image and video capture means may be any conventional still image and video capture means which include a lens or means for capturing the image of the joint, means for transmitting the captured image and means for recording and displaying said captured image. The means for capturing the image such as a lens may be located at the distal end 7 of the semi-flexible examination tube 3. In a preferred embodiment, the optical fibers are housed within tubular casing.

In a preferred embodiment, the flexible arthroscope 1 has an opening at the proximal end of the semi-flexible examination tube 3 for liquid infusion and aspiration, a passageway, and a corresponding opening at the distal end of the semi-flexible examination tube 3 to allow the user to infuse or aspirate liquid from the joint. The user may infuse or aspirate liquid from the opening in the proximal end of the semi-flexible examination tube 3 such that the liquid would travel along the passageway along the length of the semi-flexible examination tube 3 to enter or exit the opening in the distal end of the semi-flexible examination tube 3. The semi flexible examination tube 3 may have at least one channel or passageway along the length of its lumen and may have as many channels as the diameter of the semi flexible examination tube 3 will allow through which these needs are met which include but are not limited to lighting means, inflow, outflow and instrument passage and manipulation.

In the present invention, the semi-flexible examination tube 3 is removably connected to a handheld module 9 at its proximal end 11. Preferably, the semi-flexible examination tube 3 may be removably connected to a handheld module 9 via a rotary locking means. Said rotary locking means allows the semi-flexible examination tube 3 to rotate freely relative to the handheld module 9 until secured or locked into place by the user. The ability of the semi-flexible examination tube 3 to rotate freely relative to the handheld module 9 allows the user to alter the position and rotation of the handheld module 9 without altering the positioning of the distal end 7 of the semi-flexible examination tube 3 in the joint. The rotating locking means may be but is not limited to a ridge or channel on the handheld module 9 that corresponds to a lip on the proximal end of the semi-flexible examination tube 3 wherein the semi-flexible examination tube 3 may be locked or secured into place through any conventional locking means including a cam, latch or interference fit. Any conventional rotating locking means may be employed. Alternatively, the examination tube 3 may be removably connected to a handheld module 9 via any conventional locking means, including but not limited to a luer lock, snap lock, and interference fit.

The handheld module 9 of the present invention may contain the main control actuation means for the flexible arthroscope 1. The handheld module 9 may have depressible buttons for actuating the still image and video capture of the camera. The handheld module 9 may further have a valve for regulating inflow and outflow of the liquid.

FIG. 1 further illustrates the cross section of the semi-flexible examination tube 3. The semi-flexible examination tube 3 may house other channels to incorporate the needs of arthroscopic surgery including but not limited to: passageway for liquid infusion and aspiration or suction to retrieve tissue specimens or loose bodies; optical fibers or a light source; still image and video capture means; and a utility port wherein flexible instruments and materials can be introduced into the joint and wherein intra-articular tissue can be retrieved.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

We claim:

1. A flexible arthroscope for examining an internal musculoskeletal joint, which comprises:
a semi flexible examination tube having a distal end and a proximal end;
a handheld module removably connected to said proximal end of said semi flexible examination tube;
means for bending distal end of said semi flexible examination tube;
lighting means positioned in distal end of said semi flexible examination tube for illuminating said internal structure of said body; and
still image and video capture means positioned in distal end of said semi flexible examination tube for generating a visual display of said illuminated structure of said body.

2. The flexible arthroscope according to claim 1, wherein the means for bending are intercalated segments.

3. The flexible arthroscope according to claim 1, wherein the means for bending are linked segments.

4. The flexible arthroscope according to claim 2, wherein the means for bending are coupled to a pulley system to control the bending of the distal end of said semi flexible examination tube.

5. The flexible arthroscope according to claim 1, wherein the semi flexible examination tube is removably connected to said handheld module by rotating locking means.

6. The flexible arthroscope according to claim 1, wherein the semi flexible examination tube has an opening for liquid infusion and aspiration, a passageway, and a corresponding opening at the distal end of the semi-flexible examination tube.

7. The flexible arthroscope according to claim 1, wherein the semi flexible examination tube has at least one passageway.

8. An improved flexible arthroscope for examining an internal musculoskeletal joint, which comprises:
a semi flexible examination tube having a distal end and a proximal end;
a handheld module removably connected to said proximal end of said semi flexible examination tube; and
means for bending distal end of said semi flexible examination tube.

9. The improved flexible arthroscope according to claim 8, wherein the means for bending are intercalated segments.

10. The improved flexible arthroscope according to claim 8, wherein the means for bending are linked segments.

11. The improved flexible arthroscope according to claim 9, wherein the means for bending are coupled to a pulley system to control the bending of the distal end of said semi flexible examination tube.

12. The improved flexible arthroscope according to claim 8, wherein the semi flexible examination tube is removably connected to said handheld module by rotating locking means.

13. The improved flexible arthroscope according to claim 8, wherein the semi flexible examination tube has an opening for liquid infusion and aspiration, a passageway, and a corresponding opening at the distal end of the semi flexible examination tube.

14. The improved flexible arthroscope according to claim 8, wherein the semi flexible examination tube has at least one passageway.
